(19) United States
(12) Patent Application Publication
(10) Pub. No.: US 2020/0288844 A1
(43) Pub. Date: Sep. 17, 2020

(54) Beard Grooming Applicator System, Device and Beard Conditioning Formulation

(71) Applicant: Jeffrey H. Diamond, Delray Beach, FL (US)

(72) Inventor: Jeffrey H. Diamond, Delray Beach, FL (US)

(21) Appl. No.: 16/659,453

(22) Filed: Oct. 21, 2019

Related U.S. Application Data
(60) Provisional application No. 62/748,766, filed on Oct. 22, 2018.

(51) Int. Cl. A45D 34/04; A46B 9/02; A61K 8/92; A61K 8/81; A61K 8/31; A61K 8/67; A61Q 5/12

(52) U.S. Cl. CPC A45D 34/046; A46B 9/023; A46B 9/021; A61K 8/922; A61K 8/8117; A61K 8/8111; A46B 2200/1046; A61K 8/678; A61K 8/8147; A61Q 5/12; A45D 2200/25; A46B 2200/104; A61K 8/31

(57) ABSTRACT

A beard grooming system for men utilizing a device holding both a beard conditioning formulation and an application assembly permitting the beard conditioning formulation to be applied to the beard with no mess. The beard conditioning formulation utilizes various vegetable oils including Jojoba oil and copolymers to produce surface tension of the beard conditioning formulation on an applicator, such as a brush, to prevent dripping.

This application claims priority from U.S. provisional patent application Ser. No. 62/748,766 filed Oct. 22, 2018, the entire disclosure of which is incorporated herein by reference.

### BACKGROUND OF THE INVENTION

### Field of the Invention

The present invention relates to grooming of men's beards and, more particularly, to a totally self-contained beard grooming system for applying a beard conditioning formulation to the beard without requiring cleanup of the beard conditioning formulation from surfaces including the hands. More particularly, the present invention involves a system, a device and a beard conditioning formulation by which a man can comb and/or brush, clean, condition, style or otherwise groom his beard with the use of one device without requiring use of the hands to apply the beard conditioning formulation to the beard.

### Brief Discussion of the Related Art

The beard grooming and conditioning methods in the prior art are so messy that they necessitate the process be performed over a sink, preferably at home. If a man would like to groom his beard at home or away from home, on-the-go, the methods in the prior art necessitate that he bring with him a comb and/or brush and beard conditioning products such as a beard oil, a beard balm, and/or a beard serum. It would be very impractical for a man to bring with him all of the aforementioned items and to use these in a proper manner when he is on the go. The process is messy when the man is in his home. The cumbersome and messy method required to properly groom and condition a beard is one reason more men with beards do not currently use beard conditioning products.

Conditioners are commonly used for the hair since the rough texture of the hair cuticle causes the hair shaft to appear dull, dry, coarse, frizzy and unattractive. A conditioner will coat the cuticle with a hydrophobic coating that will give it a smooth texture, imparting light reflectivity to make the hair shaft appear shinier and more attractive. Beard oils in the prior art are applied to the hands, the hands are rubbed together, smearing the oil evenly over the palms and fingers, and then the hands wipe much of the oil from the hands onto the beard. Oil will always be left on the hands, and this requires an additional step of washing the hands with an appropriate detergent or emulsifier to remove oil from the hands.

Beard conditioning products typically include: beard oil, which is of very low viscosity and very runny, to condition the beard and is applied to the beard with hands or sprayed onto the beard; beard lotion, which is emulsified beard oil, to give a creamier feel upon application and has a much higher % wt solids than beard oil and less conditioning properties than beard oil; beard serum, which is beard oil in combination with vitamins and other ingredients, to support claims such as ‘helps grow beard hair faster’, it being noted that the word ‘serum’ has no definitive chemical meaning and is a word used to suggest a medicinal quality for a cosmetic product, such as ‘Skin Anti-Aging Serum’; and beard balm, which is usually compounded with waxes and high molecular weight hydrocarbons to be applied for styling and control of beard hair. Pomades are not designed to condition beard hair even though sales literature may make such a claim and are primarily used for beard styling.

Some prior art beard conditioning methods apply beard oil with an eye dropper directly to the beard or use a plastic rod to apply beard oil to the hands to be then rubbed into the beard. In other methods, oil is dispensed from a spray bottle, or a pump bottle. The above methods require that the oil is transferred from a container to the hands and then applied to the beard and massaged throughout the beard with the hands. All of the aforementioned prior art methods are messy and inefficient.

In prior art methods of applying oil directly to beards, the challenge has been to get the beard oil to stay on the beard and not drip off before the oil is distributed through the beard with the hands. These methods create additional problems of dripping oil onto the surface one is working from, which is usually the bathroom sink and, thus, have the disadvantages of getting beard oil on the counter top (that will need to be cleaned), getting the beard oil all over the hands when the oil is distributed through the beard and not applying all of the beard oil to the beard essentially wasting up to 60% of the beard oil. Applying beard conditioning product—has been very messy regardless of what prior art method is used.

Once the beard oil has been distributed as much as possible with the hands, a comb and/or brush is needed to completely distribute the beard oil through the beard.

As noted above, the prior art methods for distributing beard conditioning products (oil, balm, serum, lotion) throughout the beard are very messy. Beard conditioning products have the disadvantages of being inherently messy to apply both at home and on the go and of requiring separate application of each product. Because of these disadvantages, the methods described in the prior art make it unthinkable to employ any of these methods outside of the home in locations including, but not limited to, offices, restaurants, or any public areas.

The prior art simply shows bottles of conditioning beard oils, balms, serums, lotions, combs and brushes as the tools used to condition and groom the beard. Combs and brushes are bulky and inconvenient to carry. Beard oils, creams, balms, serums and lotions are applied by hand, which make a mess. Conditioning beard oils by nature have a very low viscosity and have a tendency to flow due to gravity and centrifugal force. This creates a constant propensity to drip, thus creating a mess when applied. The low viscosity of beard oil is due to low solids content and low molecular weight species in the oil. Additionally, the esters and fatty acids that comprise most beard oils are of low molecular weight from a polymeric perspective. In general, the molecular species that make up the various vegetable oils used in beard oils have few side chains or branching. The advantage of a beard oil formulation over a high solids formulation, such as a pomade, cream or lotion, is that the low molecular weight species in beard oils will absorb into the beard at a much greater rate producing greater beard hair conditioning efficacy. Pomades have wax, or similar high molecular weight species to facilitate styling.

To understand the requirements for growing beards for men, it is important to understand the differences between the types of facial hair, particularly beard hair and eyelash hair. The major difference between beard hair and eyelash hair is the thickness, rigidity, non-linearity and coarseness that beard hair has because it is androgenic. Androgenic hair starts forming during puberty in males. This classification of hair has the properties of being thick, rigid, and has a non-linear hair shaft giving the hair a frizzy nature. This is in diametric contrast to non-androgenic hair, such as eyelash hair or the hair on the top of the head. Non-androgenic hair is relatively thin, and flexible, especially in comparison to beard hair.

There are many conditioning products formulated especially for beards such as beard oil, beard shampoo and beard balm. This raises the question as to why beard hair should be treated with different products than other head hair. Unlike the hair on the top of the head, i.e. the scalp, and as well as eyelashes, the hair on the face is a secondary sex characteristic in men, and males begin to grow hair on their faces during adolescence. Different parts of the body sprout different types of hair: lanugo hair is the thick covering of fine hair which covers most newborn babies and falls out soon before or just after birth; vellus hair is short, fine, and lightly colored and develops all over the body during childhood, with the exception of the lips, palms, soles, back of the ears, in the navel, on genital mucosa, and in scar tissue, this hair sprouts from follicles which are not connected to an oil gland; terminal hair grows on the top of the head, it is long, thick, and darker than hair elsewhere on the body. When a person enters puberty, terminal hair replaces vellus hair in the pubic area and in the armpits. Men, in particular, may also develop terminal hair in place of vellus hair on the chest, limbs, feet, back and face. When the hair develops during puberty/adolescence, it is called androgenic hair. Androgenic hair growth is dependent on the hormone testosterone—the more testosterone a man has, the more facial hair he will grow. Ironically, testosterone is also responsible for some terminal hair follicles reverting over time to vellus follicles—hence middle-aged scalp baldness (even in the presence of a full and manly beard).

The hair that men grow naturally on their faces is very different from the hair that grows on the scalp, or elsewhere on the body, such as eyelashes. Facial hair tends to be thicker, and of a much more wiry texture than that on the scalp. The skin under beard hair is also different than the skin of the scalp. Scalp skin tends to be more oily than facial skin so using some cleansers might be great for removing excess oil from the head but too harsh and drying for the face. There are also differences in the ability of beard hair to tangle and look unruly. Scalp hair may be straight, yet beard hairs grow thicker and curly due to the shape of the follicles. Follicles on the face are much more sensitive to androgens like testosterone; these hormones make follicles twist and their resultant hairs become kinked. Thus, the brushes should be different—a stiff brush for the beard, a softer brush for the head, and an even softer brush for eyelashes. Beard hairs grow in a completely different texture than scalp hair—and even in a different color. It's not uncommon to grow red hairs in the beard when there is not a red hair to be seen elsewhere on the body. A man can retain a full head of dark hair into middle age, yet his beard may grow completely grey.

Hair is a protein filament that grows from follicles found in the dermis. Hair is one of the defining characteristics of mammals. The human body, apart from areas of glabrous skin, is covered in follicles which produce thick terminal and fine vellus hair. Most common interest in hair is focused on hair growth, hair types and hair care, but hair is also an important biomaterial primarily composed of protein, notably alpha-keratin.

Each strand of hair is made up of the medulla (innermost layer of hair shaft) and cortex. The cortex of the hair shaft is located between the hair cuticle and medulla and is the thickest hair layer. It also contains most of the hair's pigments, giving the hair its color. The pigment in the cortex is melanin which is also found in skin. The distribution of this pigment varies from animal to animal and person to person. In humans, the melanin is primarily denser nearer the medulla and cuticle. The hair cuticle is the outermost part of the hair shaft. It is formed from dead cells, overlapping in layers, which form scales that gives the hair shaft strength and provide protection for it. Although the cuticle is the outermost layer of the hair, it does not give the hair its color because it has no melanin, which is the pigment responsible for the color of a person's hair and depends on what type of melanin they have, which is found in the cortex. The inner most region, the medulla, is not always present and is an open, unstructured region. The highly structural and organized cortex, or middle layer of the hair, is the primary source of mechanical strength and water uptake. The cortex contains melanin, which colors the fiber based on the number, distribution and types of melanin granules. The shape of the follicle determines the shape of the cortex, and the shape of the fiber is related to how straight or curly the hair is. People with straight hair have round hair fibers. Oval and other shaped fibers are generally more wavy or curly. The cuticle is the outer covering. Its complex structure slides as the hair swells and is covered with a single molecular layer of lipid that makes the hair repel water. The diameter of human hair varies from 0.017 to 0.18 millimeters (0.00067 to 0.00709 in). There are two million small, tubular glands and sweat glands that produce watery fluids that cool the body by evaporation. The glands at the opening of the hair produce a fatty secretion that lubricates the hair. Hair growth begins inside the hair follicle. The only “living” portion of the hair is found in the follicle. The hair that is visible is the hair shaft, which exhibits no biochemical activity and is considered “dead”. The base of a hair's root (the “bulb”) contains the cells that produce the hair shaft. Other structures of the hair follicle include the oil producing sebaceous gland which lubricates the hair and the arréctor pili muscles, which are responsible for causing hairs to stand up. In humans with little body hair, the effect results in goose bumps.

An eyelash, or simply lash, is one of the hairs that grows at the edge of the eyelid. Eyelashes protect the eye from debris and are sensitive to being touched, thus providing a warning that an object (such as an insect) is near the eye (which then closes reflexively). The eyelashes of the embryo develop from the ectoderm between the 22nd and 26th week of pregnancy. This gives a good perspective on the delicate nature of eyelash hair. Eyelashes take about seven to eight weeks to grow back if pulled out, but constant pulling may lead to permanent damage. Their color may differ from that of the hair, although they tend to be dark on someone with dark hair and lighter on someone with light hair. Eyelash hair is not androgenic and is therefore not affected by puberty.

From the above, it should be appreciated that the beard needs to be cared for differently than scalp hair or eyelash hair. Beards tend to need a milder cleanser and more conditioning products to soften them (such as a beard oil or balm). They also benefit from specific grooming tools, like a stiff brush or comb.

The bristles of eye lash brushes are very soft by design. They are generally made of very soft extruded polymers such as silicone. The purpose of the eyelash brush is to apply cosmetic color, in solid form, to eyelash hairs. U.S. Pat. No. 8,679,467 to Cirile describes using an eyelash brush to apply a high solids, dyeless all-day coloring formula to facial hair where the formula has similar properties to mascara commonly used by women to enhance eyelashes. There are a number of products that use a method of brushing beard coloring unto a beard. Examples include “Just For Men” which uses a dye and “Blackbeard” which uses a colorant having high solids content (as a percent by weight) to apply coloration to hair, such as carbon black. This presents numerous problems as explained in U.S. Pat. No. 8,679,467 to Cirile at column 1, line 39-column 2, line 48.

The above discussion of the nature of hair is provided to enhance understanding of the present invention and the disadvantages of the prior art.

### SUMMARY OF THE INVENTION

The present invention relates to a system by which a man is enabled to groom his beard, such as by combing, brushing, cleaning and/or conditioning with the use of one device and without requiring the use of his hands to massage various products into his beard. The present invention facilitates grooming a beard when a man is at home or “on-the-go,” and away from home or the office. There has long been a need for an easy, quick, and neat method to do this at home and, especially, away from home.

The present invention uses a brush and/or comb and/or sponge-like applicator to compress and comb or brush beard hairs into desired patterns while simultaneously applying a desired amount of beard conditioning formulation.

In one aspect of the present invention, a beard conditioning product or formulation to be applied to the beard is provided in a container, such as a bottle, prior to being applied and is predominately formed of efficacious vegetable conditioning oils with no fillers or additives that slow the absorption rate of the oils into the beard hair shaft other than copolymers. The beard conditioning formulation employs low viscosity oils but the surface tension of the beard conditioning formulation is altered as well as the rheologic properties causing an increase in surface tension that prevents dripping. The beard conditioning formulation is predominately (at least 50% by weight) formed of various vegetable oils and, preferably, contains no ingredients for coloring the beard.

In accordance with the present invention, a device for use in beard grooming for men includes an applicator assembly having an applicator end, in the form of a comb and/or brush and/or foam body, contained within a bottle that has an attachable cap. The cap is connected to the applicator and holds the applicator assembly in place. The cap also functions as a handle for the applicator as well as creating a secure closure for the bottle keeping the beard conditioning formulation contained within the bottle. When the cap is removed from the bottle, the applicator end will be coated with a thin film of the beard conditioning formulation for conditioning the beard and underlying skin. The bottle opening or neck preferably carries a wiper formed integrally with the bottle or as a separate, removable gasket-like wiper. The wiper controls the amount of beard conditioning formulation that remains on the applicator end when the applicator assembly is removed from the bottle.

An additional aspect of the present invention includes a method by which a user can press the applicator end against the inside of the wiper upon removal of the applicator assembly from the bottle which will remove excess beard conditioning formulation from the applicator end. Thus, the user can control the amount of beard conditioning formulation to be applied to the beard. This may vary depending on the area of the beard one is applying the beard conditioning formulation to and the user's personal preference.

There has been a need for methods and devices that allow men to comb, brush, condition and style or groom beards, either at home or on-the-go, by using only one device and no additional products for conditioning. The present invention is a unique delivery system and method that functions as a beard grooming system. One aspect of the present invention is an applicator in the form of a comb and/or brush and/or foam (sponge) specifically designed for beards to manage the coarse nature of beard hair and penetrate the beard hair to apply a beard conditioning formula to the entire length of the beard hair shaft and provided in a bottle-type container. It is noted that combs/brushes used for other types of hair (described above), such as eyelashes, are made of very soft, highly flexible polymers such as silicon and are ineffective for use on beards.

The present invention uses a unique beard conditioning formulation which maximizes surface tension dynamics such that the formulation will immerse the applicator and will flow off the applicator onto the beard hair shafts, all the while maintaining rheologic properties that will prevent the beard conditioning formulation from dripping from the applicator and creating a clean-up challenge. The beard conditioning formulation of the present invention is designed to condition and moisturize the beard hair shaft, seal the beard hair shaft with hydrophobic hydrocarbons, infuse the beard hair shaft with low molecular weight esters and fatty acids and texturize the beard hair shaft, thus conditioning the beard hair shaft. In addition, the beard conditioning formulation of the present invention possesses physical rheologic properties such that the formulation will not drip from the applicator. Accordingly, the beard conditioning formulation of the present invention has properties preventing it from dripping, such that the beard conditioning formulation can be transferred from the applicator to the beard when desired to do so, and not before and not wasting any of the beard conditioning formula. This enables the beard conditioning formulation to transfer from the applicator to the beard hair at the time of application in one step. Additionally, the beard conditioning formulation will spread easily through the beard hair as well as to the skin underlying the beard. The present invention is formulated to have an efficacious beard conditioning utility. It is not designed to create a cosmetic tactile difference without structurally changing the beard hair shaft. Silicates can make hair feel smoother, but do not fortify the hair, and are merely cosmetic.

In the past, thickeners (materials that increase viscosity) have been added to cosmetic formulations for various reasons. A unique feature of the present invention is that copolymers in the beard conditioning formula are not for the purpose of increasing viscosity but, rather, for the purpose of increasing surface tension. The increased surface tension of the beard conditioning formula enables the transference of the beard conditioning formula from the bottle to the applicator, and from the applicator to the beard hair, in accordance with the present invention, without dripping. In other words, overcoming the force of gravity.

The grooming device of the present invention includes a container forming an internal reservoir for the beard conditioning formulation, and an elongate applicator assembly having a handle and acting as a removable cap that functions as a closure to contain the beard conditioning formulation in the reservoir of the bottle and to keep the applicator assembly securely in place allowing the applicator end of the applicator assembly to be sufficiently immersed in the beard conditioning formulation. The applicator end can include a brush and/or comb and/or a foam body. The comb has teeth of molded plastic, wood or metal of varying length and thickness. The brush has rigid bristles of plastic, a polymer, natural animal hair, such as boar, horse or other animal hair, wood or metal. The brush fibers (bristles) can be rigid and of varying length, thickness and density, depending on personal preference. The brush fibers and rigid comb teeth filaments can be any length, but normally will have a length of ¼ inch to 1 inch. The cap is connected to the applicator and acts as a handle for the applicator. The device can contain approximately 0.30 fl oz but that can change depending on the volume of the reservoir. The device reservoir can hold 2.0 fl oz for long term use.

The beard conditioning formulation, which includes hydrophobic vegetable oils, as a beard hair and underlying skin conditioner along with appropriate chemically engineered ingredients (polymers) that impart rheological properties, such as increasing cohesiveness and surface tension of the beard conditioning formulation while enhancing flow and hair shaft coating for beard conditioning. This allows the beard conditioning formulation to be used with the grooming device of the present invention. By increasing the surface tension, the flow due to gravity of the beard conditioning formulation is reduced, thereby eliminating dripping of the beard conditioning formulation off of the applicator and providing ease of application to beard hair. The copolymers in the beard conditioning formulation create a molecular “net” at the outermost surface of the beard conditioning formulation (i.e., where the beard conditioning formulation meets the air). The beard conditioning formulation includes vegetable oils with saturated and unsaturated hydrocarbon chains of varying length, in addition to fatty acid esters, organic acids, triglycerides and related hydrophobic compounds that absorb into the hair shaft and protect and seal the cuticle. Other essential oils can be used as a part of the beard conditioning formulation. The oils listed in the examples below were selected based on their absorption adequacy and the rate of absorption. This is attributed to the organic acids, esters, fatty acids, hydrophobic hydrocarbon side chains present in each oil. Accordingly, the present invention avoids leaving the beard feeling oily after the beard conditioning formulation application. The beard conditioning formula is preferably anhydrous but can be in an emulsion form containing a percentage of water.

Other aspects and advantages of the present invention will become apparent from the following description of the preferred embodiments taken in conjunction with the accompanying drawings wherein like parts in each of the several figures are identified by the same reference characters.

### BRIEF DESCRIPTION OF THE DRAWINGS

FIG. 1 is a longitudinal section of a device for grooming men's beards according to the present invention.

FIG. 2 is an exploded view of the device for grooming men's beards shown in FIG. 1.

FIGS. 3 and 4 are broken side and end views, respectively, of the applicator assembly of the device shown in FIG. 1 where the applicator end is a brush.

FIG. 5 is a broken side view of the applicator assembly of the device shown in FIG. 1 where the applicator end is a comb.

FIGS. 6 and 7 are broken side and end views, respectively, of the applicator assembly of the device shown in FIG. 1 where the applicator end is in the form of a brush and comb combination.

FIGS. 8 and 9 are broken side and end views, respectively, of the applicator assembly of the device shown in FIG. 1 where the applicator end is in the form of a comb and foam body combination.

FIGS. 10 and 11 are broken side and end views, respectively, of the applicator assembly of the device shown in FIG. 1 where the applicator end is in the form of a brush and foam body.

FIG. 12 is a broken side view of the applicator assembly of the device of FIG. 1 horizontally disposed with a brush at the applicator end carrying the beard conditioning formulation of the present invention.

### DETAILED DESCRIPTION OF THE INVENTION

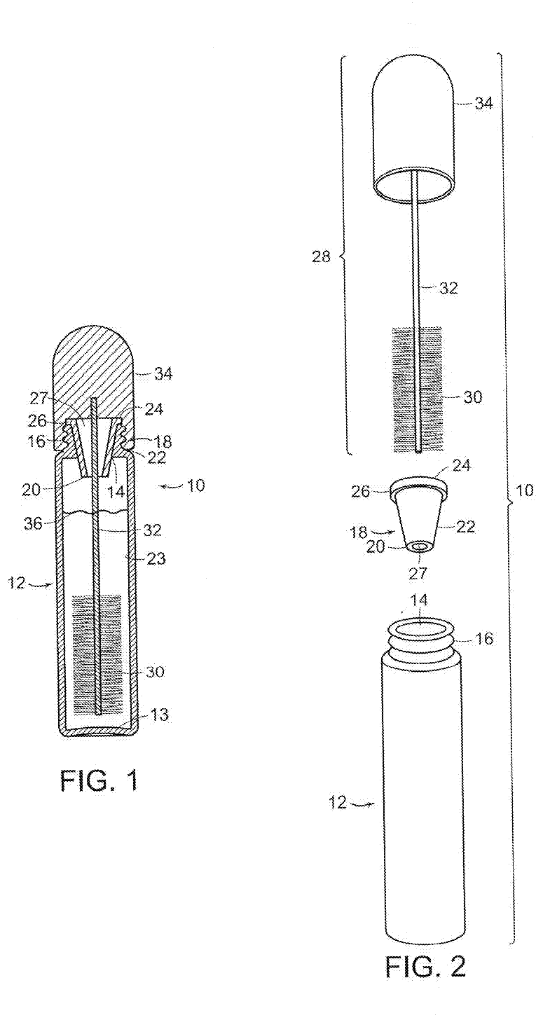

As shown in FIGS. 1 and 2, the beard grooming device 10 of the present invention includes an elongate hollow container 12, similar to a bottle, having a closed bottom end 13 and an open upper end 14 defined by a neck 16. A wiper 18 is disposed in the open end at the neck either as a separate element or integrally as part of the container. The wiper has a frustoconical shape with a smaller lower end 20 of a side wall 22 being closer to a reservoir 23 defined within the container than a larger upper end 24. The upper end has an annular flange 26 engaging the neck of the container. The wiper has a passage 27 therethrough between the lower and upper ends. An applicator assembly 28 has an applicator end 30 disposed within the container and has a shaft 32 extending from a cap or handle 34 through the wiper 18 to terminate at the applicator end 30 of the applicator assembly. The applicator end can be in the form of a brush having rigid bristles and/or a comb having rigid teeth and/or a foam body. When the beard grooming device is not in use, the applicator assembly is in the storage position shown in FIG. 1 with the handle or cap 34 secured by threaded engagement with the upper end 14 of the container such that the applicator end 30 of the applicator assembly is disposed within the container and exposed to the beard conditioning formulation 36 which is stored in the reservoir defined by the container. The device can have dimensions to allow the device to be carried in a pocket.

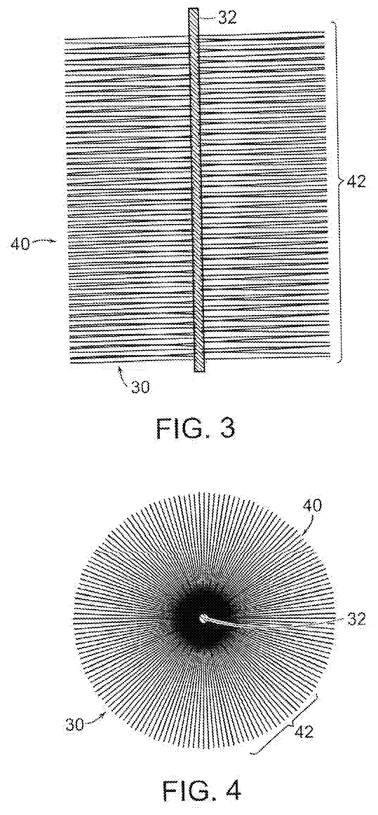

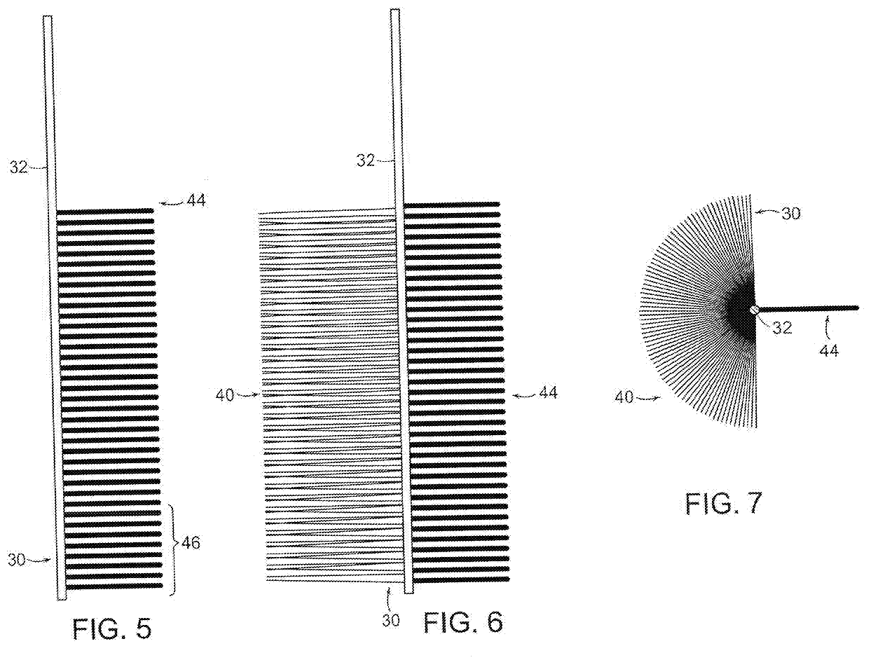

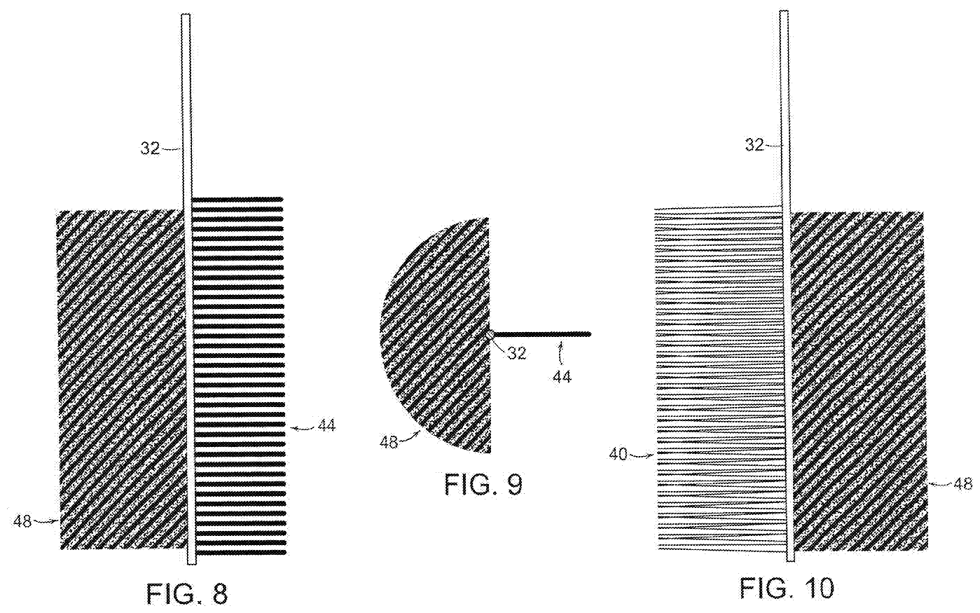

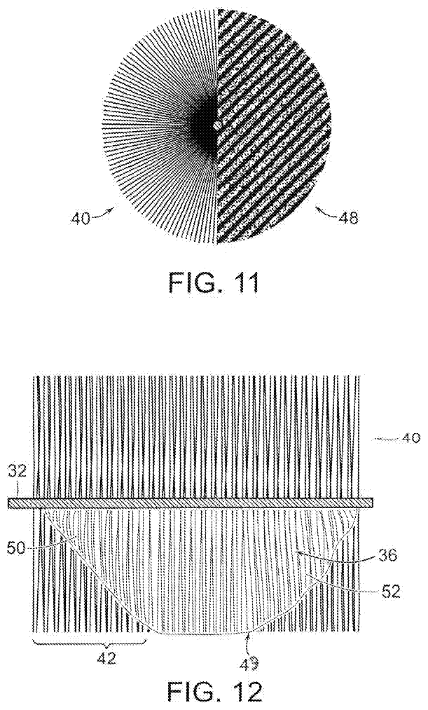

The applicator end 30 can include a brush 40 as shown in the side and end views of FIGS. 3 and 4, respectively, with the brush 40 extending along the shaft 32 such that rigid bristles 42 of the brush extend radially from the shaft. The applicator end 30 can also include a comb 44 as shown in the side view of FIG. 5 with the comb 44 extending along the shaft 32 such that rigid teeth 46 of the comb extend radially from the shaft. The applicator end 30 can also include both a brush 40 and a comb 44 on opposing sides as shown in the side and end views of FIGS. 6 and 7, respectively. The applicator end 30 shown in side and end views in FIGS. 8 and 9, respectively, combines comb 44 and foam body 48 on opposing sides of shaft 32. The applicator end 30 shown in side and end views in FIGS. 10 and 11, respectively, combines brush 40 and foam body 48 on opposing sides of shaft 32.

As shown in FIG. 12, when the applicator end is in the form of brush 40 and the brush is removed from the reservoir 23 and held in a horizontal position, the beard conditioning formulation 36 is prevented from gravitational dripping due to the surface tension and adhesion of the beard conditioning formulation. That is, gravitational and surface tension equilibrium is achieved, and the beard conditioning formulation will not drip before or during application to the beard. Copolymers in the beard conditioning formula effectively create a molecular net at the outermost surface 49 of the beard conditioning formulation 36 where the beard conditioning formulation meets the air, i.e. is exposed to the atmosphere. It has been observed that the beard conditioning formulation at the ends of the brush tapers from the shaft 32 to the tips of the bristles 42 as depicted at 50 and 52.

The beard grooming device, without the chemically engineered beard conditioning formulation according to the present invention, would not work as intended for beard grooming since it would not have the rheological properties to impregnate the applicator end or stay on the applicator end once the applicator end is immersed in the beard conditioning formulation and then removed. Without the beard conditioning formulation according to the present invention, when the applicator assembly is removed from the reservoir, virtually all of the oils would drip off of the applicator end, and there would be virtually no formulation on the applicator end.

The beard conditioning formulation of the present invention is illustrated in the following, non-limiting examples with percentage ranges for each ingredient and a specific percentage:

### Anhydrous Beard Conditioning Formula Example

| Ingredient | Range | Preferred % |
|---|---|---|
| Hydrogenated Polydecene | 1% to 15% | 10% |
| Jojoba oil | 15% to 80% | 73.8% |
| Ethylene/Propylene/Styrene | 1% to 20% | 2.5% |
| copolymer and Butylene/ | | |
| Ethylene/Styrene copolymer | | |
| Vitamin E (tocopheryl acetate) | 0.05% to 2.0% | 0.1% |
| D-Limonene | 0.05% to 3.0% | 0.2% |
| Macadamia Seed Oil | 0.1% to 4.5% | 2.4% |
| Sunflower Oil | 0.5% to 6.5% | 2.0% |
| Grape Seed Oil | 0.5% to 8.5% | 4.0% |
| | | 100% |

The above is a sample beard conditioning formulation and can be varied. For example, sunflower oil could be replaced with coconut oil. The essential oil of blend could be altered with an aromatic essential oil to achieve desired fragrance.

Other oils that are more volatile aromatic essential oils could be added from 1%-15% to modify the fragrance without interfering with the conditioning properties in a perceptible manner since most essential oils have emollient properties to a degree.

The Hydrogenated Polydecene can be Silkflo 364 marketed by Vantage Specialty Chemicals, Inc.

The Jojoba oil and the Ethylene/Propylene/Styrene copolymer and Butylene/Ethylene/Styrene copolymer can be Jojoba Glaze® LV marketed by Vantage Specialty Chemicals, Inc.

### Emulsion Beard Conditioning Formulation Example

| Ingredient | Range | Preferred % |
|---|---|---|
| Water | 30% to 60% | 50% |
| Jojoba Oil | 15% to 45% | 20% |
| Macadamia Seed Oil | 2% to 8% | 4% |
| Sunflower Oil | 2% to 8% | 4% |
| PEG 60 Hydrogenated | 5% to 15% | 8% |
| Caster Oil Trideceth -6 | | |
| Acrylates/C10-30 Alkyl | 1% to 5% | 2% |
| Acrylate Crosspolymer | | |
| Ethylene/Propylene/Styrene | 1% to 5% | 1.5% |
| Copolymer and Butylene/ | | |
| Ethylene/Styrene Copolymer | | |
| Tocopherol Acetate | 0.1% to .2% | 0.1% |
| D-Limone | .1% to .4% | 0.2% |
| Denatured Alcohol | 3.0% to 10% | 5.0% |
| Benzyl Alcohol | 3% to 15% | 5.2% |
| | | 100% |

As noted, the first example described above is an anhydrous (without water) beard conditioning formulation example used in conjunction with the applicator according to the present invention. This beard conditioning formulation is close to 100% vegetable oil. The second example described above is an emulsion. There are several emulsifiers effective to bridge the oil and water phase of the beard conditioning formulation. Some examples include but are not limited to: PEG-30 Dipolyhydroxystearate, PEG-60 Hydrogenated Castor Oil, and Trideceth-6.

In order to prevent the emulsified oil and water beard conditioning formulation from dripping off of the applicator, beard conditioning formulation viscosity is altered. One polymer that will alter the viscosity is Acrylates/C10-30 Alkyl Acrylate Crosspolymer. This polymer used at, or around, 3% wt/wt will increases the viscosity of the beard conditioning formulation and prevents the beard conditioning formulation dripping off the applicator.

Adjusting surface tension, much more so than adjusting viscosity, is a critical key in enabling the beard conditioning formulation to work properly in accordance with the present invention. Many cosmetic formulations have a formulation that increases viscosity. Viscosity is a measure of flow. Higher viscosity, in and of itself, will not impede the beard conditioning formulation from dripping from the applicator. Higher viscosity does not create higher surface tension. Surface tension and viscosity are independent variables, and often their relationship is counter-intuitive.

Viscosity is adjusted in cosmetics to alter sensorial perception, and spreadability. Adjusting rheologic properties has not been a focus in cosmetic additives.

Acrylates/C10-30 Alkyl Acrylate Crosspolymer. (Pemulen™ TR-1*) polymer is a high molecular weight, crosslinked copolymer of acrylic acid and a hydrophobic C10-30 alkyl acrylate co-monomer. It is a versatile, non-ethoxylated polymer designed to create stable and mild oil-in-water emulsions at low use levels. In most cases, the oils in the beard conditioning formulation will be <30% wt/wt. This polymer is added to the emulsion beard conditioning formulation example in order to increase the viscosity of the water phase of the emulsion. The ethylene/propylene/styrene and butylene/ethylene/styrene copolymers are used to increase the surface tension through the oil phase.

The method of use of the present invention is simple:
1. Unscrew cap.
2. Remove applicator.
3. A small amount of the beard conditioning formulation is automatically applied to the applicator.
4. Comb or brush one side of the beard including mustache, and then repeat on the other side of beard. Brush one side of the beard going with the grain of the hair growth. Then brush the beard against the grain in order to spread the conditioning formulation. Finally brush beard and mustache with the beard grain. Repeat on other side of beard if more beard conditioning formulation is desired.
5. If more beard conditioning formulation is desired, repeat the above steps.
6. Insert applicator into bottle and screw cap on tightly.

The best time to use the present invention is after washing the face or showering. An advantage of the present invention, compared to the prior art, is that it enables one to brush and condition the beard in locations where the process would have been virtually impossible to carry out using the prior art. The present invention can be used after a shower, after a coffee break, after cigarette or cigar, lunch or dinner.

The device is designed to apply a set amount of beard conditioning formulation to the applicator. If it is desired to remove some of the beard conditioning formulation, simply wipe the applicator end against the inside of the wiper structure. The wiper structure separates the outside of the bottle orifice from the inside of the bottle. Almost all the beard conditioning formulation can be removed from the brush if desired. If it is desired to apply more beard conditioning formulation to an area of the beard, simply give that area two applications.

The present invention enables the user to brush and condition the beard with one product, mess free. This exhibits tremendous utility and improvement over any beard conditioning method in the prior art since the hands are not required to contact the beard conditioning formulation or the beard to apply the beard conditioning formulation to condition and groom the beard.

The beard conditioning formulation saturated applicator end can be pressed against the beard to transfer the conditioning formulation from the applicator to the beard. After the beard conditioning formulation has been transferred to the beard, the brush or comb can be used to work the beard conditioning formulation throughout the beard for example, by stroking the beard from the top down and from the bottom up to distribute the beard conditioning formulation throughout beard.

The beard conditioning formulation of the present invention is designed to be efficacious as a beard and underlying skin moisturizer, emollient, hydrophobic hair sealant and conditioner. Prior art products to color beards include talc powder, black coal, iron oxide tan and/or iron oxide brown which are the antithesis of the conditioning properties of the beard conditioning formulation of the present invention since the more inert solids that are added to a formulation, the less beard hair conditioning properties the formulation will possess, if any at all.

As explained above, the present intention fills the need for a method to apply beard conditioners (oils) to the beard in a mess free manner in that it enables the user to brush beard conditioning oils into the beard without oil touching the hands and to simultaneously comb and/or brush the beard hair. The modified, highly efficacious, essential oil beard conditioning formulation has rheologic properties that enable it to remain on the applicator and at the same time be able to be transferred to beard hairs. With the use of specific copolymers from a polymer class, the viscosity will increase and reduce the flow of the “liquid.” Additionally, the copolymers increase the surface tension at the beard conditioning formulation. The copolymers comprise at least one alkene monomer. In particular, the monomer is selected from monomers of ethylene, propylene, butylene, and styrene. It is noted that an alkene monomer is a hydrocarbon monomer with a linear or branched carbon-based chain that contains a carbon-carbon double bond. A copolymer is a polymer comprising two different monomers. Such copolymers have good compatibility with hydrocarbons and good adhesion to keratin fibers such as beard hair. Additionally, the copolymer of the beard conditioning formulation has good adhesion to the fibers of the applicator brush. The copolymer is supplied by Vantage Specialty Chemicals, Inc. under the tradename of Jojoba Glaze. The copolymer has a molecular mass in Daltons greater than 1000. Preferably greater than 2000 to 150,000.

With the conditioning oils modified by the copolymers into the beard conditioning formulation of the present invention, unique rheological properties are created such that the oil formulation component of the present invention functions effectively and enables the method of the present invention to condition the beard and underlying skin.

The primary elements of fluid dynamics for the beard conditioning formulation to be used with the grooming device 10 are cohesion, which causes water, oil and other liquids to form drops, surface tension which causes the drops to be nearly spherical, and stay encapsulated, and adhesion keeps the drops in place. Cohesive forces among liquid molecules are responsible for the phenomenon of surface tension. The molecules well inside the liquid are attracted equally in all directions by the other similar molecules. The molecules on the surface (molecules at the liquid/air interface) experience an inward pull. Surface tension is the energy required to increase the surface area of a liquid by a given amount. The stronger the intermolecular interactions, the greater the surface tension.

Liquids that are non-polar, such as mixtures of vegetable oils, have little molecular electrostatic bonding, and as a result have very little cohesive forces within the liquid mass. The copolymer in the beard conditioning formulation of the present invention increases the cohesive forces of the beard conditioning formulation, and as a result, alter the physical properties of the liquid's outer membrane with an elastic, net-like polymeric film. The beard conditioning formulation of the present invention is engineered so that the outer membrane (or the liquid/air interface) has elasticity in order to absorb the surface tension, as well as the stress and strain the outer membrane must withstand in order for the formulation to stay on the applicator all the way from the liquid in the bottle, through the air, to the beard hair shaft outer surface or cuticle.

Adhesive forces are the acting forces between the molecules of different types and are a negative factor relative to the prior art method of applying beard oils to the beard by applying the oil with the palms of the hands used as an applicator. Adhesive forces are forces of attraction between a liquid and a solid surface. The difference in strength between cohesive forces and adhesive forces determine the behavior of a liquid in contact with a solid surface. Adhesive forces keep the beard conditioning formulation of the present invention bound to the applicator, while cohesive forces and surface tension keep the beard conditioning formulation as an encapsulated mass. The polymers used in the beard conditioning formulation of the present invention have innate ability to increase the surface tension of the beard conditioning formulation and as a result cause the beard conditioning formulation to stay encapsulated and on the applicator. This enables the beard conditioning formulation to work in conjunction with the applicator device 10 to provide a method for applying a beard conditioning formulation to the beard hair. Other polymers such as Acylates/10-30 Alkyl Acrylate Cross polymer, functions as a thickening agent (viscosity increasing agent), texture enhancer, film-forming agent, and an emulsifier.

Viscosity, unlike surface tension relates to movement. An important differentiating point between surface tension and viscosity is the fact that, while one would think that thick fluids would translate to a high surface tension, and that thin fluids would produce lower surface tension, this is not the case.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A beard grooming system for men comprising
a container having an open end and defining a reservoir for receiving a beard conditioning formulation;
an elongate applicator assembly carried by said container, said applicator assembly having a handle end, an applicator end and a shaft extending between said handle end and said applicator end;
a wiper disposed adjacent said open end and of said container and having a passage therethrough slidably receiving said shaft of said applicator assembly;
said applicator assembly having a storage position with said handle end closing said open end of said container and said applicator end disposed in said reservoir and immersed in said beard conditioning formulation to be coated with said beard conditioning formulation, said applicator assembly being removable from said storage position by sliding said shaft and said applicator end through said wiper passage to wipe excess beard conditioning formulation from said applicator end; and
said beard conditioning formulation clinging to said applicator end to resist dripping due to surface tension of said beard conditioning formulation when said applicator assembly is removed from said storage position.

2. A beard grooming system as recited in claim 1 wherein said applicator end includes a brush extending along said shaft and having rigid bristles extending radially from said shaft.

3. A beard grooming system as recited in claim 1 wherein said applicator end includes a comb formed of rigid teeth extending radially from said shaft.

4. A beard grooming system as recited in claim 1 wherein said applicator end includes a foam body.

5. A beard grooming system as recited in claim 2 wherein said brush is disposed on a first side of said shaft and said applicator end includes a comb formed of rigid teeth extending radially from said shaft opposite said first side.

6. A beard conditioning formulation comprising
various vegetable oils, including jojoba oil constituting at least 50% of the beard conditioning formulation by weight;
ethylene/propylene/styrene copolymers; and
butylene/ethylene/styrene copolymers.

7. A beard conditioning formulation as recited in claim 6 wherein said vegetable oils constitute at least 60% of the beard conditioning formulation by weight.

8. A beard conditioning formulation as recited in claim 7 wherein said copolymers constitute up to 10% of the beard conditioning formulation by weight.

9. A beard conditioning formulation as recited in claim 6 wherein said vegetable oils include Macadamia seed oil, 2.4% of the beard conditioning formulation by weight, Sunflower oil, 2.0% of the beard conditioning formulation by weight, and Grape Seed oil, 4.0% of the beard conditioning formulation by weight, and Jojoba oil, 78.8% of the beard conditioning formulation by weight.

10. A beard conditioning formulation as recited in claim 9 wherein said beard conditioning formulation includes Hydrogenated Polydecene, 10% of the beard conditioning formulation by weight, Vitamin E, 0.1% of the beard conditioning formulation by weight and D-Limonene, 0.2% of the beard conditioning formulation by weight.

11. A beard conditioning formulation as recited in claim 6 wherein said beard conditioning formulation is an emulsion and includes Acrylates/C10-30 Alkyl Acrylate Crosspolymer as an emulsifier.

12. A beard conditioning formulation as recited in claim 5 wherein said beard conditioning formulation is anhydrous.

13. A beard conditioning formulation as recited in claim 1 wherein said beard conditioning formulation is an emulsion.
